SEQUENCE LISTING (34 sequences)

SEQ ID NO: 1 - DNA, 19 bp, synthetic construct
  source 1..19: mol_type = other DNA
acaaggctgccccgactac

SEQ ID NO: 2 - DNA, 20 bp, synthetic construct
  source 1..20: mol_type = other DNA
tgggctcataccagggtttg

SEQ ID NO: 3 - DNA, 19 bp, synthetic construct
  source 1..19: mol_type = other DNA
ctttcccgtggaccttcca

SEQ ID NO: 4 - DNA, 21 bp, synthetic construct
  source 1..21: mol_type = other DNA
ctcggagcctgtagtgcagtt

SEQ ID NO: 5 - DNA, 20 bp, synthetic construct
  source 1..20: mol_type = other DNA
tgtcagtgcctgcagaccat

SEQ ID NO: 6 - DNA, 20 bp, synthetic construct
  source 1..20: mol_type = other DNA
caagggagcttcagggtcaa

SEQ ID NO: 7 - DNA, 19 bp, synthetic construct
  source 1..19: mol_type = other DNA
ggctgttgtggccagtgaa

SEQ ID NO: 8 - DNA, 21 bp, synthetic construct
  source 1..21: mol_type = other DNA
actttttgaccgcccttgaga

SEQ ID NO: 9 - DNA, 21 bp, synthetic construct
  source 1..21: mol_type = other DNA
ctcatcgtggtgggtaacgtg

SEQ ID NO: 10 - DNA, 21 bp, synthetic construct
  source 1..21: mol_type = other DNA
acacacagcacatctaccgaa

SEQ ID NO: 11 - DNA, 20 bp, synthetic construct
  source 1..20: mol_type = other DNA
gtgccacccacaagaaagct

SEQ ID NO: 12 - DNA, 20 bp, synthetic construct
  source 1..20: mol_type = other DNA
cctggaagacccgggaatag

SEQ ID NO: 13 - DNA, 19 bp, synthetic construct
  source 1..19: mol_type = other DNA
ggccctctctagttcccag

SEQ ID NO: 14 - DNA, 21 bp, synthetic construct
  source 1..21: mol_type = other DNA
tagccatcaaacctgttgagc

SEQ ID NO: 15 - DNA, 20 bp, synthetic construct
  source 1..20: mol_type = other DNA
gccaaggacgcagctttcta

SEQ ID NO: 16 - DNA, 23 bp, synthetic construct
  source 1..23: mol_type = other DNA
ttcagggacagagtctccttctg

SEQ ID NO: 17 - DNA, 23 bp, synthetic construct
  source 1..23: mol_type = other DNA
ggagacttggcatgacctagaag

SEQ ID NO: 18 - DNA, 22 bp, synthetic construct
  source 1..22: mol_type = other DNA
atgtcacagcggtgtagaggaa

SEQ ID NO: 19 - DNA, 21 bp, synthetic construct
  source 1..21: mol_type = other DNA
tcgcatcgacacagatgagaa

SEQ ID NO: 20 - DNA, 19 bp, synthetic construct
  source 1..19: mol_type = other DNA
ggctgtcgccgtagtacca

SEQ ID NO: 21 - DNA, 23 bp, synthetic construct
  source 1..23: mol_type = other DNA
gcacagttggcaacctttatgaa

SEQ ID NO: 22 - DNA, 24 bp, synthetic construct
  source 1..24: mol_type = other DNA
ctcatgacaggcaaagatcaactt

SEQ ID NO: 23 - DNA, 21 bp, synthetic construct
  source 1..21: mol_type = other DNA
acggccaggtcatcactattg

SEQ ID NO: 24 - DNA, 24 bp, synthetic construct
  source 1..24: mol_type = other DNA
agaggtctttacggatgtcaacgt

SEQ ID NO: 25 - DNA, 23 bp, synthetic construct
  source 1..23: mol_type = other DNA
tgtagcccatgttgtagcaaacc

SEQ ID NO: 26 - DNA, 24 bp, synthetic construct
  source 1..24: mol_type = other DNA
gaggacctgggagtagatgaggta

SEQ ID NO: 27 - DNA, 22 bp, synthetic construct
  source 1..22: mol_type = other DNA
ctgagcaccttctttcccttca

SEQ ID NO: 28 - DNA, 21 bp, synthetic construct
  source 1..21: mol_type = other DNA
tggaccagacatcaccaagct

SEQ ID NO: 29 - DNA, 21 bp, synthetic construct
  source 1..21: mol_type = other DNA
gggaattcaccccaagaacat

SEQ ID NO: 30 - DNA, 23 bp, synthetic construct
  source 1..23: mol_type = other DNA
gtcactgttcagcatcttttcga

SEQ ID NO: 31 - DNA, 18 bp, synthetic construct
  source 1..18: mol_type = other DNA
gcccctggccactgaact

SEQ ID NO: 32 - DNA, 21 bp, synthetic construct
  source 1..21: mol_type = other DNA
caagctttctgcccattcttg

SEQ ID NO: 33 - DNA, 21 bp, synthetic construct
  source 1..21: mol_type = other DNA
catgttcgtcatgggtgtgaa

SEQ ID NO: 34 - DNA, 21 bp, synthetic construct
  source 1..21: mol_type = other DNA
ggcatggactgtggtcatgag

(19) United States
(12) Patent Application Publication
(10) Pub. No.: US 2026/0256718 A1
(43) Pub. Date: Sep. 3, 2026

(54) USE OF AGENT TARGETING NOREPINEPHRINE-ADRENERGIC RECEPTOR BETA 2 (NE-Adrb2) SIGNALING PATHWAY IN PREPARATION OF DRUG FOR TREATING DRY EYE DISEASE (DED)

(71) Applicant: EYE INSTITUTE OF SHANDONG FIRST MEDICAL UNIVERSITY, Qingdao (CN)

(72) Inventors: Qingjun ZHOU, Qingdao (CN); Mingli QU, Qingdao (CN); Lixin XIE, Qingdao (CN); Xiaofei BAI, Qingdao (CN)

(73) Assignee: EYE INSTITUTE OF SHANDONG FIRST MEDICAL UNIVERSITY, Qingdao (CN)

(21) Appl. No.: 19/318,902

(22) Filed: Sep. 4, 2025

(30) Foreign Application Priority Data
Feb. 28, 2025 (CN) 202510238060.5

(51) Int. Cl. A61K 31/138; A61K 9/00; A61K 31/137; A61P 27/02

(52) U.S. Cl. CPC A61K 31/138; A61K 9/0048; A61K 31/137; A61P 27/02

(57) ABSTRACT

Use of an agent targeting a norepinephrine-adrenergic receptor beta 2 (NE-Adrb2) signaling pathway in preparation of a drug for treating dry eye disease (DED) is provided, belonging to the technical field of DED therapeutics. Use of an agent targeting an NE-Adrb2 signaling pathway in preparation of a drug for treating DED. Specifically, the agent targeting the NE-Adrb2 signaling pathway includes an agent that blocks the NE-Adrb2 signaling pathway. It is found that the NE-Adrb2 signaling pathway directly contributes to the pathogenesis of DED, and targeting the NE-Adrb2 signaling pathway enables the development of therapeutic agents for DED. In particular, an adrenergic receptor beta 2 (Adrb2) blocker can alleviate the severity of DED, thereby achieving therapeutic efficacy. This discovery provides a novel therapeutic strategy for the prevention and treatment of DED.

### CROSS REFERENCE TO RELATED APPLICATION

This patent application claims the benefit and priority of Chinese Patent Application No. 202510238060.5 filed with the China National Intellectual Property Administration on Feb. 28, 2025, the disclosure of which is incorporated by reference herein in its entirety as part of the present application.

### REFERENCE TO SEQUENCE LISTING

A computer readable XML file entitled “SEQUENCE LISTING”, that was created on Jul. 25, 2025, with a file size of about 30896 bytes, contains the sequence listing for this application, has been filed with this application, and is hereby incorporated by reference in its entirety.

### TECHNICAL FIELD

The present disclosure belongs to the technical field of dry eye disease (DED) therapeutics, and relates to use of an agent targeting a norepinephrine-adrenergic receptor beta 2 (NE-Adrb2) signaling pathway in preparation of a drug for treating DED.

### BACKGROUND

Dry eye disease (DED) is a chronic, multifactorial disorder primarily affecting the tear film and ocular surface, resulting in symptoms including ocular pain, burning sensation, and visual disturbance^{[1-3]}. The pathogenesis of DED involves tear film instability and elevated osmolarity, which cause progressive damage to the ocular surface^{[4]}. Among various etiological factors, chronic stress (encompassing environmental and psychological stress) plays a critical role in the initiation and progression of DED by disrupting ocular surface homeostasis and exacerbating local inflammatory responses^{[5-8]}. Studies have demonstrated that chronic stress also activates the sympathetic nervous system (SNS)^{[9,10]}. However, the relationship between SNS and DED pathogenesis remains incompletely understood.

The cornea is predominantly innervated by sensory nerves from the ophthalmic branch of the trigeminal ganglion^{[11]}. However, recent studies have revealed sympathetic nerve distribution originating from the superior cervical ganglion (SCG) within the cornea^{[12-14]}. Anatomical evidence indicates that sympathetic nerves are primarily localized in the limbal region, serving as a barrier between the vascularized conjunctiva and avascular corneal tissue^{[12,15]}. SNS activation and norepinephrine (NE) release have been implicated in corneal pathologies including alkali burns, pathogenic infections, and neovascularization, all exacerbating corneal inflammation^{[16-18]}. Chemical sympathectomy using 6-hydroxydopamine (6-OHDA) or local NE depletion via N-(2-chloroethyl)-N-ethyl-2-bromobenzylamine hydrochloride (DSP-4) has demonstrated potential in mitigating inflammatory responses^{[12,15,19]}. NE participates in corneal pathophysiology by triggering local inflammation, delaying wound healing, activating macrophages, modulating chemokine expression, and recruiting immune cells^{[15,20,21]}. While these findings provide an important basis for sympathetic involvement in corneal disorders, significant deficiencies exist in sympathetic nerve-targeting therapeutics for DED. The unclear mechanistic relationship between SNS and DED pathogenesis has hindered development of precisely targeted drugs for DED, resulting in limited clinical DED treatment options. Consequently, there exists an urgent unmet need for novel therapeutics addressing this gap in DED management.

### CITED REFERENCES

1. Craig J P, et al. TFOS DEWS II Definition and Classification Report. Ocul Surf 2017; 15 (3): 276-283.
2. Clayton J A. Dry Eye. The New England journal of medicine. 2018; 378 (23): 2212-2223.
3. Belmonte C, et al. TFOS DEWS II pain and sensation report. The ocular surface. 2017; 15 (3): 404-437.
4. Stapleton F, et al. TFOS DEWS II Epidemiology Report. The ocular surface. 2017; 15 (3): 334-365.
5. Tiskaoglu N S, et al. Dry Eye Disease in Patients with Newly Diagnosed Depressive Disorder. Current eye research. 2017; 42 (5): 672-676.
6. Sano K, et al. Enriched environment alleviates stress-induced dry-eye through the BDNF axis. Scientific reports. 2019; 9 (1): 3422.
7. Wang S, et al. Obstructive Sleep Apnea Affects Lacrimal Gland Function. Investigative ophthalmology & visual science. 2022; 63 (3): 3.
8. Zhang S, et al. Hyperglycemia Induces Tear Reduction and Dry Eye in Diabetic Mice through the Norepinephrine-α(1) Adrenergic Receptor-Mitochondrial Impairment Axis of Lacrimal Gland. Am J Pathol. 2023; 193 (7): 913-926.
9. Ji Y W, et al. The Dopaminergic Neuronal System Regulates the Inflammatory Status of Mouse Lacrimal Glands in Dry Eye Disease. Investigative ophthalmology & visual science. 2021; 62 (4): 14.
10. Cardoso F, et al. Neuro-mesenchymal units control ILC2 and obesity via a brain-adipose circuit. Nature. 2021; 597 (7876): 410-414.
11. Yu F X, et al. The impact of sensory neuropathy and inflammation on epithelial wound healing in diabetic corneas. Progress in retinal and eye research. 2022; 89: 101039.
12. Xue Y, et al. Stress systems exacerbate the inflammatory response after corneal abrasion in sleep-deprived mice via the IL-17 signaling pathway. Mucosal Immunol. 2024; 17 (3): 323-345.
13. Yun H, et al. Sensory Nerve Retraction and Sympathetic Nerve Innervation Contribute to Immunopathology of Murine Recurrent Herpes Stromal Keratitis. Invest Ophthalmol Vis Sci. 2022; 63 (2): 4.
14. Marfurt C F, et al. Sensory and sympathetic innervation of the mammalian cornea. A retrograde tracing study. Invest Ophthalmol Vis Sci. 1989; 30 (3): 461-472.
15. Xue Y, et al. The mouse autonomic nervous system modulates inflammation and epithelial renewal after corneal abrasion through the activation of distinct local macrophages. Mucosal Immunol. 2018; 11 (5): 1496-1511.
16. Lasagni Vitar R M, et al. A Hypothalamic-Controlled Neural Reflex Promotes Corneal Inflammation. Invest Ophthalmol Vis Sci. 2021; 62 (13): 21.
17. Yun H, et al. Production of the Cytokine VEGF-A by CD4(+) T and Myeloid Cells Disrupts the Corneal Nerve Landscape and Promotes Herpes Stromal Keratitis. Immunity. 2020; 53 (5): 1050-1062.e1055.
18. Dong Q, et al. Overactivation of Norepinephrine-β2-Adrenergic Receptor Axis Promotes Corneal Neovascularization. Invest Ophthalmol Vis Sci. 2023; 64(3): 20.
19. Ma X, et al. Corneal epithelial injury-induced norepinephrine promotes Pseudomonas aeruginosa keratitis. Exp Eye Res. 2020; 195: 108048.
20. Liu J, et al. Sympathetic Signals Promote Immunosuppressive Neutrophils in Lung Tumors. bioRxiv. 2024: 2024. 2002. 2015. 580434.
21. He S, et al. Sympathetic Nerves Coordinate Corneal Epithelial Wound Healing by Controlling the Mobilization of Ly6Chi Monocytes From the Spleen to the Injured Cornea. Invest Ophthalmol Vis Sci. 2023; 64 (12): 13.

### SUMMARY

A purpose of the present disclosure is to provide use of an agent targeting an NE-Adrb2 signaling pathway in preparation of a drug for treating DED. It has been found that the NE-Adrb2 signaling pathway directly contributes to the pathogenesis of DED, and targeting the NE-Adrb2 signaling pathway enables the development of therapeutic agents for DED.

The present disclosure provides use of an agent targeting an NE-Adrb2 signaling pathway in preparation of a drug for treating DED.

Preferably, the agent targeting the NE-Adrb2 signaling pathway includes an agent that blocks the NE-Adrb2 signaling pathway.

The present disclosure further provides use of an Adrb2 blocker in preparation of a drug for treating DED.

The present disclosure further provides use of an Adrb2 blocker in preparation of a drug having at least one effect selected from the group consisting of i) to vi):
i) increasing tear secretion;
ii) improving a corneal epithelial barrier function;
iii) improving corneal innervation;
iv) reducing expression of a matrix metalloproteinase (MMP);
v) ameliorating inflammatory responses in cornea and conjunctiva; and
vi) restoring a conjunctival goblet cell density.

The present disclosure further provides use of an Adrb2 blocker in preparation of a drug for downregulating an inflammation-associated pathway.

Preferably, the Adrb2 blocker includes ICI 118551.

The present disclosure further provides use of N-(2-chloroethyl)-N-ethyl-2-bromobenzylamine hydrochloride (DSP-4) in preparation of a drug for treating DED.

The present disclosure provides use of an adrenergic receptor beta (Adrb) blocker in preparation of a drug for treating DED.

Preferably, the DED is selected from the group consisting of aqueous-deficient DED and psychological stress-associated DED.

Preferably, the drug includes a topical ophthalmic formulation.

The present disclosure provides use of an agent targeting an NE-Adrb2 signaling pathway in preparation of a drug for treating DED. Utilizing a classical murine DED model induced by chronic desiccation stress combined with scopolamine treatment, elevated SNS activation and increased corneal NE levels are observed. Topical application of NE has directly induced DED-associated inflammatory and immune responses in healthy mice, demonstrating SNS involvement in DED pathogenesis. It has been found that the NE-Adrb2 signaling pathway directly contributes to the pathogenesis of DED, and targeting the NE-Adrb2 signaling pathway enables the development of therapeutic agents for DED. Herein, blockers of the NE-Adrb2 signaling pathway including systemic SNS agents, local NE-depleting agents, and Adrb2 blockers have exhibited therapeutic efficacy on the DED. Experimental results have demonstrated that the Adrb2 blocker has significantly increased tear secretion, improved corneal epithelial barrier function, enhanced corneal innervation, reduced matrix metalloproteinase (MMP) expression, ameliorated corneal/conjunctival inflammation, and restored conjunctival goblet cell density. Moreover, the Adrb2 blocker has downregulated key inflammatory signaling pathways including TNF, NF-κB, chemokine, and IL-17. These findings establish that the Adrb2 blocker may alleviate the severity of DED, thereby achieving therapeutic efficacy. This discovery provides a novel therapeutic strategy for the prevention and treatment of DED.

### BRIEF DESCRIPTION OF THE DRAWINGS

To describe the technical solutions in examples of the present disclosure or in the prior art more clearly, the accompanying drawings required in the examples are briefly described below. Apparently, the accompanying drawings in the following description show merely some examples of the present disclosure, and other drawings can still be derived from these accompanying drawings by those of ordinary skill in the art without creative efforts.

FIGS. 1A-1F shows analysis of sympathetic activity and corneal NE levels in dry eye mice; where FIG. 1A shows tear secretion measurement after scopolamine-induced desiccation stress; FIG. 1B shows sodium fluorescein staining and scoring of corneas on day 7 post-scopolamine injection; FIG. 1C shows comparative mRNA levels of TNF-α and IL-1β in mouse corneas; FIG. ID shows representative immunofluorescence images and quantification of c-FOS+TH+ neurons in SCG 2 h post-scopolamine injection, Scale bar: 50 μm; FIG. 1E shows corneal NE content after combined treatment; FIG. 1F shows immunofluorescence staining of NE in limbal and central cornea, Scale bar: 20 μm; n=3-6 per group; data represent mean±SEM; *P<0.05, **P<0.001, ns: not significant;

FIGS. 2A-2G shows therapeutic effects of NE depletion on dry eye severity; where FIG. 2A shows experimental design schematic for 6-OHDA/DSP-4 treated mice; FIG. 2B shows corneal NE concentration on day 7 post-scopolamine modeling; FIG. 2C shows tear volume measurement for 6-OHDA/DSP-4 treated mice; FIG. 2D shows sodium fluorescein staining and scoring on day 7; FIG. 2E to FIG. 2F show representative images and quantification of MMP3, MMP9, and TUNEL+ cells in corneal epithelium, Scale bar: 20 μm; FIG. 2G shows corneal TNF-α and IL-1β mRNA levels; n=3-6 per group; data represent mean±SEM; *P<0.05, **P<0.01, ***P<0.001;

FIGS. 3A-3H shows effects of topical NE instillation on dry eye induction; where FIG. 3A shows experimental design schematic of local NE instillation; FIG. 3B shows tear secretion after NE treatment; FIG. 3C to FIG. 3D show sodium fluorescein staining and scoring on day 7; FIG. 3E shows corneal mRNA levels of TNF-α, IL-1β, Cxcl1 and Cxcl2; FIG. 3F to FIG. 3H show representative images and quantification of MMP3, MMP9, and TUNEL+ cells, Scale bar: 20 μm; n=3-6 per group; data represent mean±SEM; *P<0.05, **P<0.01, ***P<0.001, ns: not significant;

FIGS. 4A-4F shows resistance of Adrb2−/− mice to NE-induced dry eye; where FIG. 4A shows mRNA levels of adrenergic receptors in corneas; FIG. 4B shows Adrb2 mRNA expression in Adrb2−/− mice; FIG. 4C shows tear volume after 10 mM NE instillation; FIG. 4D shows sodium fluorescein scoring on day 7; FIG. 4E shows corneal mRNA levels of TNF-α, IL-1β, Cxcl1 and Cxcl2; FIG. 4F shows representative images and quantification of MMP3, MMP9 and TUNEL+ cells, Scale bar: 20 μm; n=3-6 per group; data represent mean±SEM; *P<0.05, **P<0.01, ***P<0.001;

FIGS. 5A-5K shows therapeutic effects of Adrb2 blocker on dry eye; where FIG. 5A shows experimental design for Adrb2 blocker ICI 118551 (ICI) treatment; FIG. 5B shows tear secretion in ICI-treated mice; FIG. 5C shows sodium fluorescein scoring on day 14; FIG. 5D to FIG. 5E show representative images and quantification of MMP3, MMP9 and TUNEL+ cells, Scale bar: m; FIG. 5F shows TUBB3 immunofluorescence, Scale bar: 20 μm; FIG. 5G shows corneal NGF and Sema3a mRNA levels; FIG. 5H shows corneal TNF-α and IL-1β mRNA levels; FIG. 5I to FIG. 5J show TNF-α and IL-1β protein levels in cornea/conjunctiva; FIG. 5K shows PAS staining and goblet cell density in conjunctiva, Scale bar: 50 μm; n=3-6 per group; data represent mean±SEM; *P<0.05, **P<0.01, ***P<0.001;

FIGS. 6A-6H shows anti-inflammatory mechanisms of Adrb2 blockade; where FIG. 6A shows volcano plot of differentially expressed genes in the cornea of dry eye mice treated with ICI (ICI) or solvent (vehicle); FIG. 6B shows KEGG enrichment of downregulated genes (top 10 pathways) in ICI-treated and vehicle groups; FIG. 6C shows heatmap of DEGs; FIG. 6D shows corneal mRNA levels of Cxcl1, Cxcl2, IL17c, and S100a9; FIG. 6E shows CD45 immunofluorescence in corneal sections, Scale bar: 50 μm; FIG. 6F shows representative images of NF-κB p65 nuclear translocation under: normal medium, 500 mOsM hyperosmotic medium, and 500 mOsM hyperosmotic medium with ICI, Scale bar: 20 μm; FIG. 6G shows p-NF-κB p65 protein levels; FIG. 6H shows mRNA levels of TNF-α, IL-1β, CXCL1, and CXCL2 in human corneal epithelial cells (HCECs); n=3-4 per group; data represent mean±SEM; *P<0.05, **P<0.01, ***P<0.001, ns: not significant;

FIG. 7 shows dose-dependent effects of Adrb2 blockade on tear secretion in dry eye mice;

FIG. 8 shows concentration-dependent inhibition of hyperosmolarity-induced inflammatory factors by Adrb2 blocker;

FIG. 9 shows therapeutic efficacy comparison between Adrb2 blocker and glaucoma drugs;

FIGS. 10A-10C shows restraint stress model results; where FIG. 10A shows tear secretion; FIG. 10B shows sodium fluorescein scores; FIG. 10C shows NE levels; and

FIGS. 11A-11C shows ICI 118551 (β2-AR blocker) treatment in restraint stress-induced dry eye; where FIG. 11A shows experimental design of restraint stress-induced DED; FIG. 11B shows tear volume; FIG. 11C shows relative mRNA levels of TNF-α and IL-1β.

### DETAILED DESCRIPTION OF THE EMBODIMENTS

The present disclosure provides use of an agent targeting an NE-Adrb2 signaling pathway in preparation of a drug for treating DED. In an embodiment, the agent targeting the NE-Adrb2 signaling pathway includes an agent that blocks the NE-Adrb2 signaling pathway. The agent blocking the NE-Adrb2 signaling pathway includes: systemic SNS ablation agents, local NE depletion agents, Adrb2 blockers. In particular embodiments, the local NE depletion agent is N-(2-chloroethyl)-N-ethyl-2-bromobenzylamine hydrochloride (DSP-4). The DED includes aqueous-deficient DED or psychological stress-associated DED. In an embodiment, the drug includes topical ophthalmic formulations. DED is an ocular surface inflammatory disease triggered by chronic environmental and psychological stress. Accumulating evidence indicates SNS plays a central role in chronic stress responses. Critically, the present disclosure first establishes: direct linkage between DED pathogenesis and SNS activation and specific mechanistic actions via the axis. Experimental confirmation includes: SNS hyperactivation and NE overproduction in murine DED models (chronic desiccation stress+scopolamine). Effective inflammation reduction and DED symptom alleviation through: systemic SNS ablation or local NE depletion. Key discoveries are: topical NE treatment impairs corneal barrier function, reduces tear secretion, and dose-dependently induces corneal inflammation. These findings demonstrate that chronic stress-induced SNS activation elevates corneal NE levels, directly triggering DED pathogenesis and revealing a novel mechanism of stress-induced DED. The role of NE-Adrb2 signaling axis in the pathogenesis of DED is discovered in the present disclosure. Mechanistic validation: Adrb2 blockade downregulating 208 significantly altered genes in DED corneas, with top 10 pathways enriched in inflammatory responses. In vitro confirmation: Adrb2 inhibition suppresses hyperosmolarity-induced NF-κB signaling pathway activation and proinflammatory cytokine expression in HCECs. Adrb2−/− mice exhibit resistance to NE-induced DED. Conclusion: SNS activation induces DED primarily through NE-Adrb2 signaling pathway-mediated inflammation. The activation of SNS induced by chronic stress is directly involved in the occurrence of DED. This work establishes targeting the NE-Adrb2 signaling pathway as a novel therapeutic approach for DED prevention and treatment.

The present disclosure further provides use of an Adrb2 blocker in preparation of a drug for treating DED. In a specific embodiment, the Adrb2 blocker includes ICI 118551. The DED includes aqueous-deficient DED or psychological stress-associated DED. The drug includes topical ophthalmic formulations. The experimental results show that SNS activation and its neurotransmitter NE have induced DED-related corneal inflammatory response through Adrb2 in the mouse cornea, providing a novel potential strategy for the prevention and treatment of DED.

The present disclosure further provides use of an Adrb2 blocker in preparation of a drug having at least one effect selected from the group consisting of i) to vi):
i) increasing tear secretion;
ii) improving a corneal epithelial barrier function;
iii) improving corneal innervation;
iv) reducing expression of a matrix metalloproteinase (MMP);
v) ameliorating inflammatory responses in cornea and conjunctiva; and
vi) restoring a conjunctival goblet cell density.

In a specific embodiment, the Adrb2 blocker includes ICI 118551. The drug includes topical ophthalmic formulations. Experimental results demonstrate that topical treatment with the β2-AR blocker ICI 118551 has significantly restored tear secretion, improved corneal barrier integrity, thus lowering sodium fluorescein scores. ICI 118551 markedly suppressed MMP3/MMP9 expression, reduced corneal epithelial apoptosis. ICI 118551 enhanced corneal innervation, ameliorated corneal/conjunctival inflammation, restored conjunctival goblet cell density. Collectively, these data establish the therapeutic potential of Adrb2 blockade for DED treatment.

The present disclosure further provides use of an Adrb2 blocker in preparation of a drug for downregulating an inflammation-associated pathway. In a specific embodiment, the Adrb2 blocker includes ICI 118551. The drug includes topical ophthalmic formulations. In specific embodiments, the inflammation-associated pathways include at least one selected from a group consisting of TNF, NF-κB, chemokine, and IL-17 signaling pathways. Experimental results demonstrate that ICI 118551 treatment has induced upregulation of 174 genes and downregulation of 134 genes; KEGG pathway analysis confirms significant downregulation of inflammation-associated pathways (including TNF, NF-κB, chemokine, and IL-17 signaling pathways). There is substantially reduced expression of inflammation-related genes within these pathways. These findings indicate that Adrb2 antagonism effectively regulates DED-associated inflammatory responses by downregulating key pathways such as NF-κB.

The present disclosure provides use of an adrenergic receptor beta (Adrb) blocker in preparation of a drug for treating DED. In a specific embodiment, the Adrb blocker is an Adrb2 blocker. In a specific embodiment, the Adrb2 blocker includes ICI 118551. The DED includes aqueous-deficient DED or psychological stress-associated DED.

The present disclosure further provides use of N-(2-chloroethyl)-N-ethyl-2-bromobenzylamine hydrochloride (DSP-4) in preparation of a drug for treating DED. Specifically, the DED includes aqueous-deficient DED or psychological stress-associated DED.

In a specific embodiment, the drug includes topical ophthalmic formulations. In a specific embodiment, the topical ophthalmic formulation includes an eye drop. The drug includes a medicine for subconjunctival injection.

In order to further illustrate the present disclosure, the use of an agent targeting an NE-Adrb2 signaling pathway in preparation of a drug for treating DED provided by the present disclosure will be described in detail below with reference to the examples, but they should not be construed as limiting the protection scope of the present disclosure.

### Example 1

### Animal Model

Female C57BL/6J mice aged 8-12 weeks were purchased from Vital River Laboratory Animal Technology Co., Ltd. (Beijing, China). Adrb2-knockout mice (Adrb2−/−) were generated by GemPharmatech Co., Ltd. (Nanjing, China; Strain ID: T007691).

To establish an aqueous-deficient dry eye model, experimental group mice received subcutaneous injections of scopolamine hydrobromide (0.5 mg/0.2 mL PBS, Abcam, Cat #ab141080) three times daily (08:00, 14:00, 20:00) for 7 d under desiccating stress. Control group mice remained untreated.

For systemic NE depletion, experimental group mice were intraperitoneally injected with 6-hydroxydopamine hydrobromide (6-OHDA, MCE, Cat #HY-B1081A, 80 mg/kg dissolved in 0.9% sterile saline containing 0.1% ascorbic acid). Injections were administered once daily for the first four days (from Day −6 to Day −3), followed by injections every three days (from Day −3 to Day 7), totaling six administrations.

For local NE depletion, experimental group mice received subconjunctival injections of 5 L N-(2-chloroethyl)-N-ethyl-2-bromobenzylamine hydrochloride (DSP-4, neurotoxin, MCE, Cat #HY-103210, 20 mg/mL in PBS) on Day −1 and Day 3. Control mice received equivalent volumes of 0.1% ascorbic acid-saline or PBS.

To evaluate NE's effect on dry eye induction, experimental group mice were topically administered 5 μL of 0.2-10 mM NE (MCE, Cat #HY-13715A, freshly dissolved in PBS) four times daily for 7 d, with PBS as a vehicle control.

To investigate β2-AR blocker, dry eye model mice were treated with topical ICI 118,551 (20 μM, MCE, Cat #HY-13951) four times daily for 7 consecutive days, using PBS as a vehicle control

### Tear Secretion Measurement

Tear volume was measured using phenol red-impregnated cotton threads (Jingming, Cat #30059010). Threads were placed at the inferior conjunctival fornix for 15 s, and the length of the wetted portion (in millimeters) was recorded.

### Corneal Sodium Fluorescein Staining

Corneal epithelial damage was assessed by sodium fluorescein staining. Eyes were instilled with 0.25% sodium fluorescein solution (Jingming, Tianjin, China) and examined under a slit-lamp microscope (BQ900, Haag-Streit, Bern, Switzerland). Corneas were divided into four quadrants, and staining intensity in each quadrant was scored independently according to established criteria (Qu M, et al. Therapeutic Effects of STAT3 Inhibition on Experimental Murine Dry Eye. Invest Ophthalmol Vis Sci. 2019; 60 (12): 3776-3785).

### Cell Culture and Treatment

HCECs were cultured as previously described (Qu M, et al. Different cellular effects of four anti-inflammatory eye drops on human corneal epithelial cells: independent in active components. Mol Vis. 2011; 17: 3147-3155). To establish hyperosmotic conditions, 90 mmol/L NaCl was added to the medium, achieving 500 mOsm. Cells were maintained in: isotonic medium (312 mOsM), hyperosmotic medium (500 mOsM), and 20 μM ICI 118551 (treatment group). After 12-h treatment, cells were harvested for RNA extraction and Western blot analysis.

Quantitative Real-Time PCR (qPCR)

Total RNA was extracted from mouse corneas and HCECs using TransZol™ Up Plus RNA Kit (TransGen Biotech, Beijing, China). First-strand cDNA was synthesized with Hiscript First-Strand cDNA Synthesis Kit (Vazyme, Nanjing, China). qPCR reactions were conducted using SYBR Green Master Mix (Roche, Germany) on an Applied Biosystems 7500 Real-Time PCR System (Applied Biosystems, USA). Primer sequences were listed in Table 1.

TABLE 1
Primer sequences of qPCR

| Gene | Forward primer (SEQ ID NO:) | Reverse primer (SEQ ID NO:) |
|---|---|---|
| m-TNF-α | ACAAGGCTGCCCCGACTAC (1) | TGGGCTCATACCAGGGTTTG (2) |
| m-IL-1β | CTTTCCCGTGGACCTTCCA (3) | CTCGGAGCCTGTAGTGCAGTT (4) |
| m-Cxcl1 | TGTCAGTGCCTGCAGACCAT (5) | CAAGGGAGCTTCAGGGTCAA (6) |
| m-Cxcl2 | GGCTGTTGTGGCCAGTGAA (7) | ACTTTTTGACCGCCCTTGAGA (8) |
| m-Adrb1 | CTCATCGTGGTGGGTAACGTG (9) | ACACACAGCACATCTACCGAA (10) |
| m-Adrb2 | GTGCCACCCACAAGAAAGCT (11) | CCTGGAAGACCCGGGAATAG (12) |
| m-Adrb3 | GGCCCTCTCTAGTTCCCAG (13) | TAGCCATCAAACCTGTTGAGC (14) |
| m-NGF | GCCAAGGACGCAGCTTTCTA (15) | TTCAGGGACAGAGTCTCCTTCTG (16) |
| m-Sema3a | GGAGACTTGGCATGACCTAGAAG (17) | ATGTCACAGCGGTGTAGAGGAA (18) |
| m-IL-17c | TCGCATCGACACAGATGAGAA (19) | GGCTGTCGCCGTAGTACCA (20) |
| m-S100a9 | GCACAGTTGGCAACCTTTATGAA (21) | CTCATGACAGGCAAAGATCAACTT (22) |
| m-β-actin | ACGGCCAGGTCATCACTATTG (23) | AGAGGTCTTTACGGATGTCAACGT (24) |
| h-TNF-α | TGTAGCCCATGTTGTAGCAAACC (25) | GAGGACCTGGGAGTAGATGAGGTA (26) |
| h-IL-1β | CTGAGCACCTTCTTTCCCTTCA (27) | TGGACCAGACATCACCAAGCT (28) |
| h-CXCL1 | GGGAATTCACCCCAAGAACAT (29) | GTCACTGTTCAGCATCTTTTCGA (30) |
| h-CXCL2 | GCCCCTGGCCACTGAACT (31) | CAAGCTTTCTGCCCATTCTTG (32) |
| h-GAPDH | CATGTTCGTCATGGGTGTGAA (33) | GGCATGGACTGTGGTCATGAG (34) |

### Immunofluorescence Staining

For immunofluorescence, SCG and ocular tissues were embedded in OCT compound and sectioned at 7 μm thickness. Sections were fixated with 4% paraformaldehyde (PFA) for 10 min and washed with PBS. SCG sections were permeabilized with PBS containing 3% Triton X-100 for 30 min. Both SCG and corneal sections were blocked with 5% donkey serum at room temperature for 1 h, followed by incubation with primary antibodies at 4° C. overnight. HCECs were fixated with 4% PFA for 10 min, permeabilized with 0.3% Triton X-100 for 15 min, blocked with 5% donkey serum, and incubated with primary antibodies. All samples were detected using Alexa Fluor-conjugated secondary antibodies (Invitrogen), and images were acquired with a ZEISS LSM 880 confocal microscope (Zeiss, Germany). Primary antibody details were provided in Table 2.

### TUNEL Staining

Corneal epithelial apoptosis was assessed by TUNEL assay using frozen eye sections according to the manufacturer's protocol (Roche, Cat #11 684817910). Sections were counterstained with DAPI, and images were captured using a fluorescence microscope (Nikon).

### ELISA Analysis

Corneal or conjunctival tissues from four eyes were pooled as one sample. NE (Abnova, Cat #KA3836), TNF-α (Proteintech, Cat #KE10002), and IL-1β (Proteintech, Cat #KE10003) levels were measured using ELISA kits per manufacturer's instructions. Absorbance at 450 nm was read on a microplate reader (Molecular Devices, USA), and protein concentrations were calculated against standard curves.

### Histology and Staining

Mouse eyeballs were fixed with 4% PFA, embedded in paraffin, and sectioned at 4 μm thickness. Sections underwent hematoxylin and eosin (H&E) staining and periodic acid-Schiff (PAS) staining (MXB, Fuzhou, China) following kit protocols. Representative images were captured using a light microscope (Nikon, Tokyo, Japan).

### RNA Sequencing and Data Processing

Corneal tissues from ICI 118551-treated and untreated dry eye mice were harvested 14 d after scopolamine hydrobromide injections (four corneas pooled per sample). Total RNA was extracted and libraries were constructed using the VAHTS™ Universal V6 RNA-seq Library Prep Kit (Vazyme). Sequencing was conducted on the Illumina NovaSeq 6000 platform (Illumina, San Diego, USA) by OE Biotech (Shanghai). Raw sequencing data in FASTQ format were processed with fastp to filter low-quality reads, yielding high-quality clean reads. Clean reads were aligned to the reference genome using HISAT2. Gene expression levels were quantified as FPKM (Fragments Per Kilobase of transcript per Million mapped reads), and read counts were generated by HTSeq-count. Principal Component Analysis (PCA) was conducted in R (v3.2.0) to assess sample reproducibility. Differential expression analysis used DESeq2 with thresholds of Q-value <0.05 and |fold change|>2 or <0.5 to identify significant differentially expressed genes (DEGs). KEGG pathway enrichment analysis employed hypergeometric testing in R (v3.2.0), with significance defined as p<0.05 or corrected Q<0.05.

### Western Blotting

HCECs from experimental groups were lysed in ice-cold RIPA buffer (containing protease inhibitor cocktail; Roche, Germany). Proteins were separated by SDS-PAGE and transferred to PVDF membranes. Membranes were blocked with 5% BSA for 1 h at room temperature, then incubated with primary antibodies (Table 2) overnight at 4° C. After three TBST washes, membranes were incubated with goat anti-rabbit secondary antibody (ZSGB-BIO, Beijing, China, Cat #ZDR-5306, 1:3000 dilution) for 1 h at room temperature. Protein bands were detected using chemiluminescent substrate (ECL Kit, Chemicon, Temecula, USA) and band intensity was quantified with ImageJ software (NIH, USA).

TABLE 2
Primary antibodies for immunofluorescence and Western blotting

| Primary antibodies | Manufacturer | Cat. No. | Dilution rate |
|---|---|---|---|
| TH | Merck-millipore | AB1542 | IF: 1:200 |
| c-FOS | Abcam | ab190289 | IF: 1:200 |
| NE | Sigma-Aldrich | AB120 | IF: 1:200 |
| MMP3 | Abcam | ab73955 | IF: 1:200 |
| MMP9 | Proteintech | 10375-2-AP | IF: 1:200 |
| TUBB3 | Merck-Millipore | 15708A4 | IF: 1:200 |
| CD45 | Servicebio | GB113886 | IF: 1:200 |
| NF-κB p65 | Servicebio | GB11997 | IF: 1:200 |
| NF-κB p65 | Cell Signaling | 8242 | WB: 1:500 |
| | Technology | | |
| Phospho-NF-κB p65 | Cell Signaling | 3033 | WB: 1:500 |
| | Technology | | |

IF: Immunofluorescence staining,
WB: Western blotting

### Statistical Analysis

Statistical analyses were conducted using GraphPad Prism 8.0. Comparisons between two groups used unpaired two-tailed Student's t-test; multiple group comparisons used one-way ANOVA followed by Tukey's post hoc test. Data were expressed as mean±SEM, and P<0.05 was considered statistically significant.

### Ethical Approval

All experimental protocols were approved by the Ethics Committee of Shandong Eye Institute. Procedures complied with the ARVO Statement for the Use of Animals in Ophthalmic and Vision Research.

### Chronic Dry Eye Stress Elevates SNS Activity and Corneal NE Levels

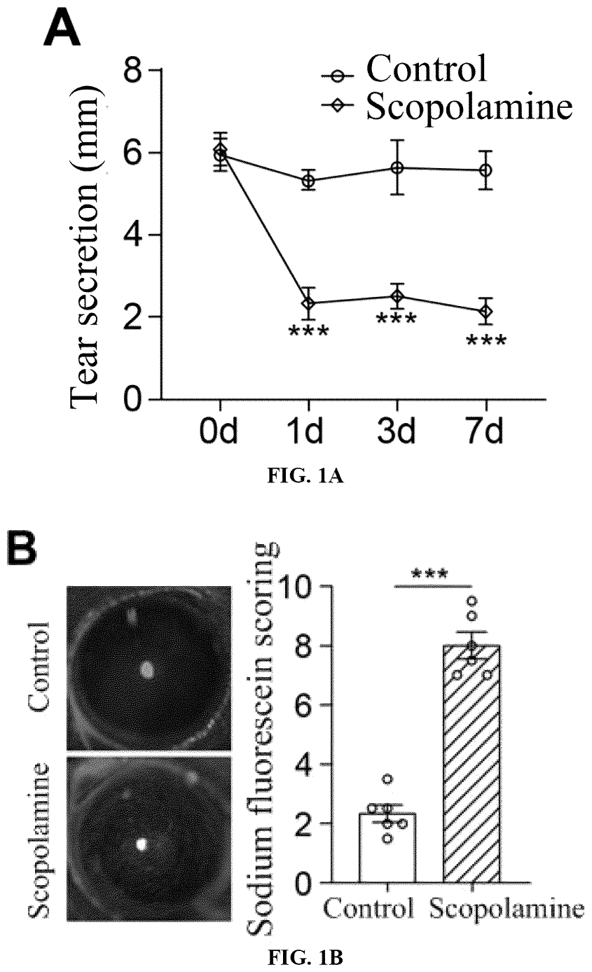

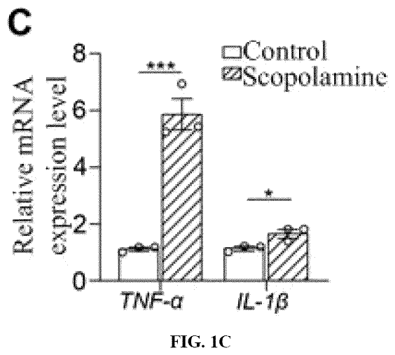

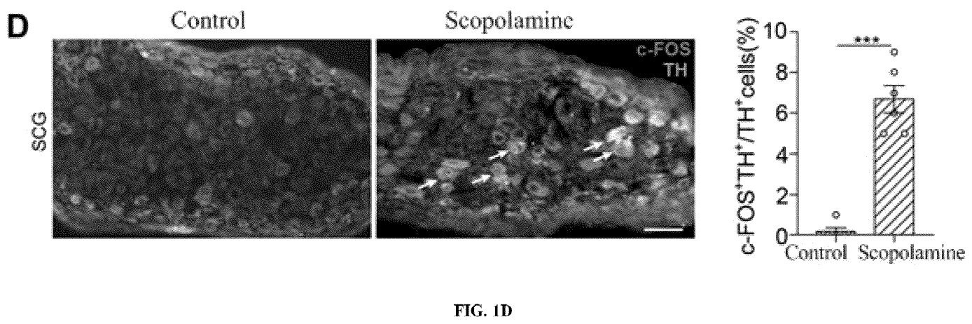

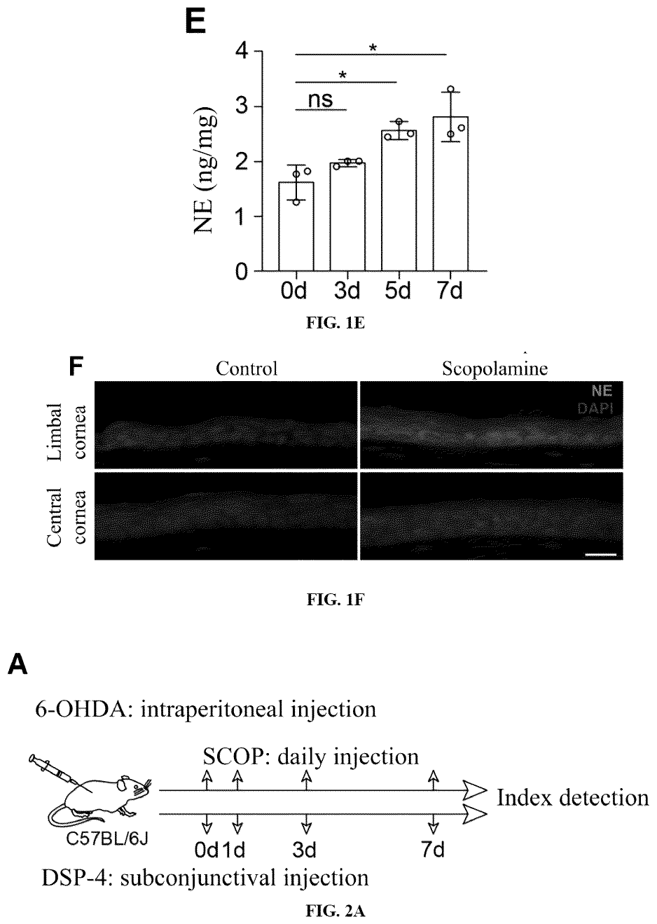

To investigate the relationship between chronic dry eye stress and SNS activation, a murine DED model was established using scopolamine combined with desiccating stress for 7 d, with age-matched healthy mice as controls. Results showed that scopolamine-treated mice exhibited rapid tear reduction, decreasing to 30% of normal levels by day 7 (FIG. 1A). Concurrently, corneal epithelial barrier function was impaired, with fluorescence scores 3.4-fold higher than controls (FIG. 1B). qPCR confirmed significant upregulation of TNF-α and IL-1β mRNA in DED corneas (FIG. 1C). Furthermore, the number of c-FOS+TH+ neurons in the SCG increased markedly in DED mice (FIG. 1D). ELISA revealed significantly elevated corneal norepinephrine (NE) levels in DED mice from day 5 post-scopolamine treatment (FIG. 1E). Immunofluorescence demonstrated that elevated NE localized primarily to limbal and central corneal epithelial regions of DED mice, with higher intensity in limbal areas (FIG. 1F). Collectively, these findings indicated that dry eye stress induced SNS activation and elevated corneal NE levels, suggesting SNS involvement in DED pathogenesis.

### Systemic and Local NE Depletion Alleviating Dry Eye Severity

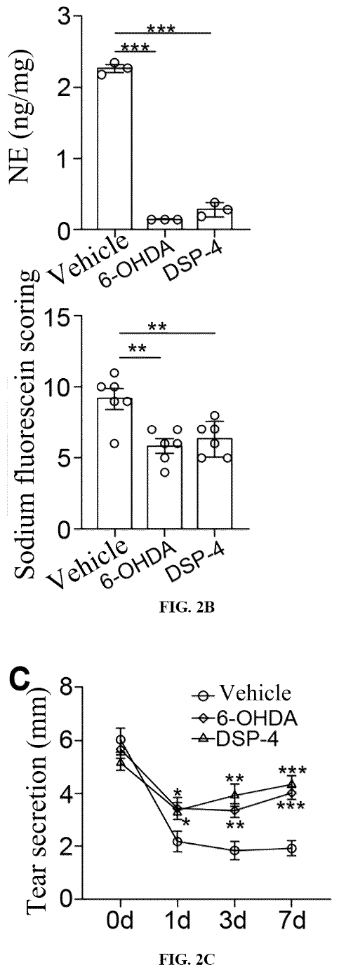

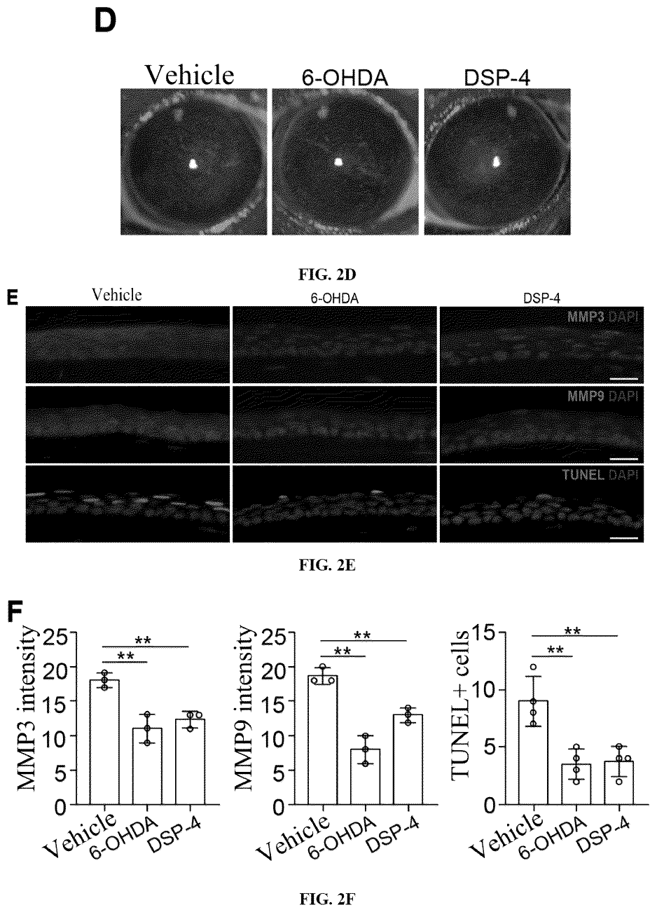

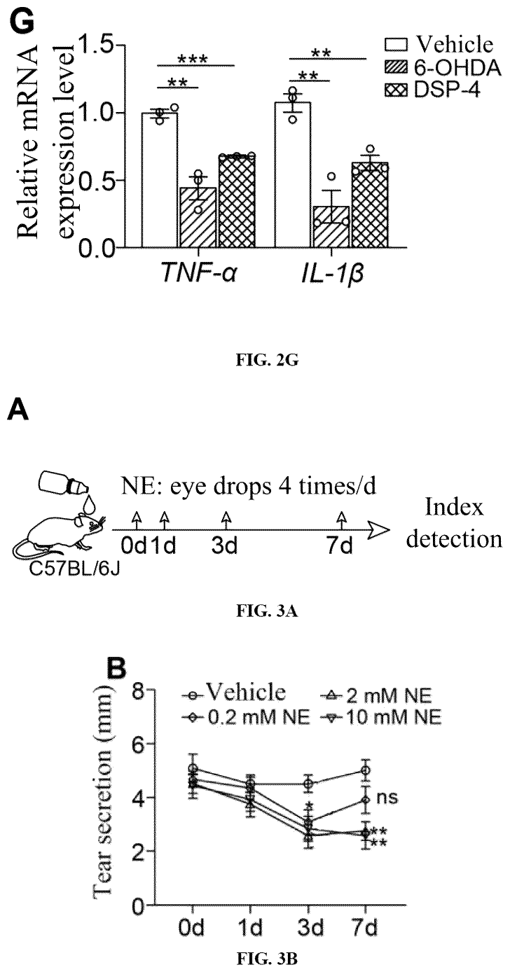

To investigate sympathetic activation in DED pathogenesis, systemic SNS ablation and local NE depletion were conducted in scopolamine-induced dry eye mice. Systemic SNS ablation was achieved via intraperitoneal injection of the selective neurotoxin 6-OHDA, whereas local NE depletion used subconjunctival injection of DSP-4 (FIG. 2A). Systemic SNS ablation significantly reduced corneal NE levels in DED mice (FIG. 2B). Concurrently, tear secretion increased 2-fold in sympathectomized DED mice versus controls by day 7 (FIG. 2C). Keratopathy improvement manifested as: reduced corneal fluorescein scores (FIG. 2D), decreased MMP3/MMP9 expression, diminished epithelial apoptosis (FIG. 2E and FIG. 2F), and downregulated TNF-α, and IL-1β mRNA (FIG. 2G). Similar therapeutic effects, such as increased tear volume and ameliorated corneal damage, were observed in DED mice following local NE depletion with DSP-4 (FIG. 2B to FIG. 2G). These results demonstrated that both systemic SNS ablation and local NE depletion effectively mitigated DED severity, confirming elevated NE as pivotal in DED pathogenesis.

### Topical NE Application Directly Inducing DED Symptoms

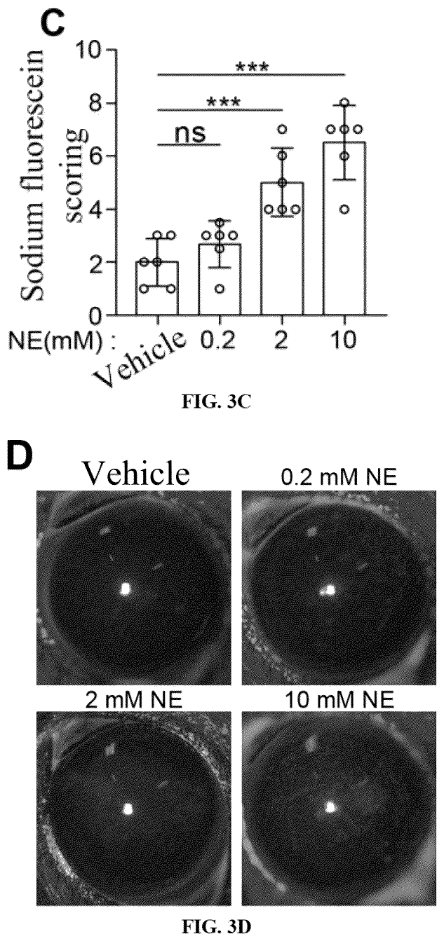

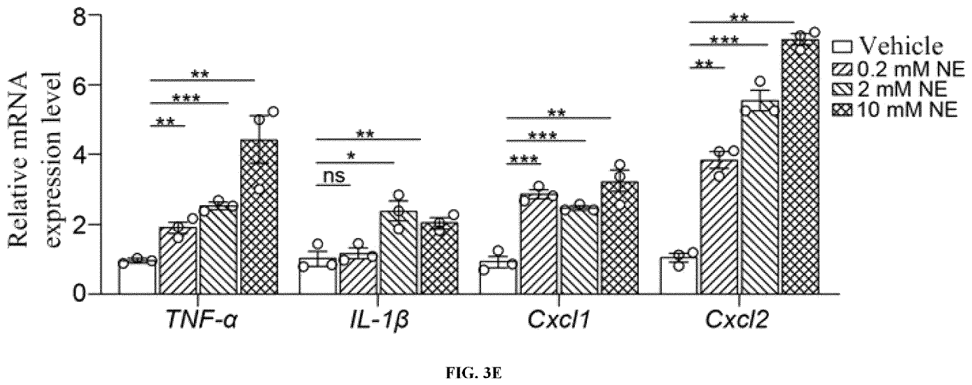

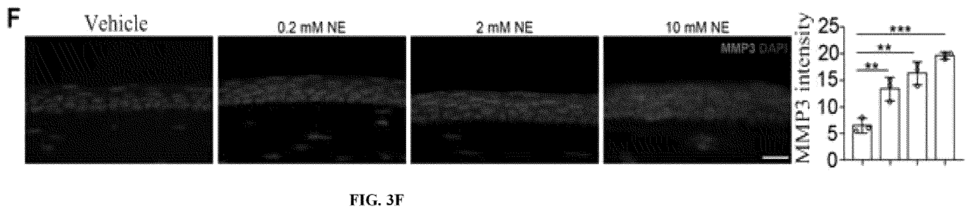

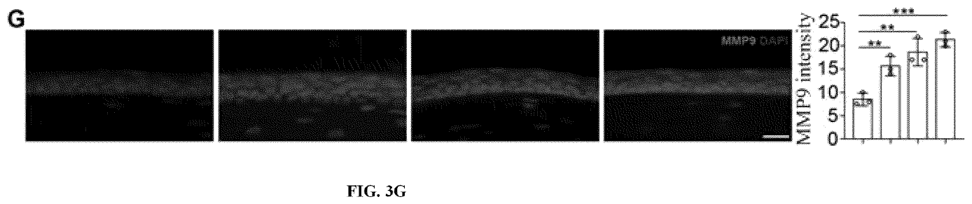

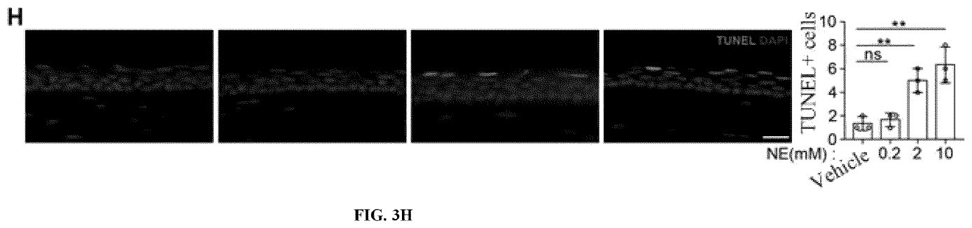

To examine NE's pathogenic role in DED, healthy mice received topical NE (0.2-10 mM) 4 times daily for 7 d (FIG. 3A). All NE-treated groups exhibited reduced tear secretion, with higher concentrations causing greater suppression, indicating concentration-dependent inhibition (FIG. 3B). By day 7, tear volume decreased to 61% (2 mM NE) and 58% (10 mM NE) of control levels. Conversely, 0.2 mM NE caused transient reduction (day 3) but full recovery by day 7. Sodium fluorescein scoring showed no difference between 0.2 mM NE and controls, whereas 2 mM and 10 mM NE groups had significantly higher scores (FIG. 3C and FIG. 3D). qPCR revealed dose-dependent upregulation of TNF-α, Cxcl1, and Cxcl2 mRNA in all NE groups, while IL-1β increased only with 2 mM and 10 mM NE (FIG. 3E). Immunofluorescence confirmed concentration-dependent elevation of MMP3/MMP9 in corneal epithelium (FIG. 3F and FIG. 3G). TUNEL staining demonstrated apoptosis in 2 mM/10 mM NE groups but not in 0.2 mM NE or controls (FIG. 3H). These data established that NE could directly induce DED-like symptoms in mice, further confirming the role of NE in the DED pathogenesis.

### Adrb2 Gene Knockout Blocking NE-Induced DED Symptoms

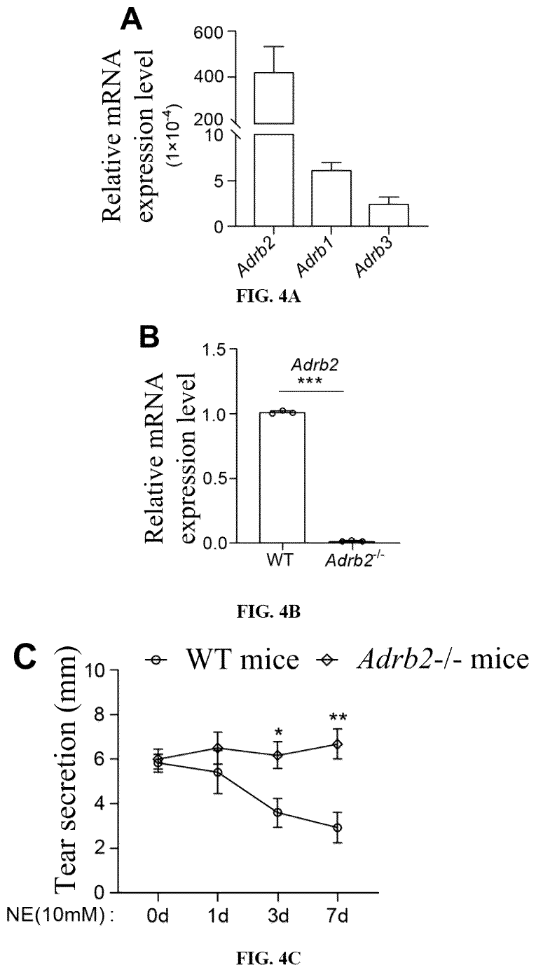

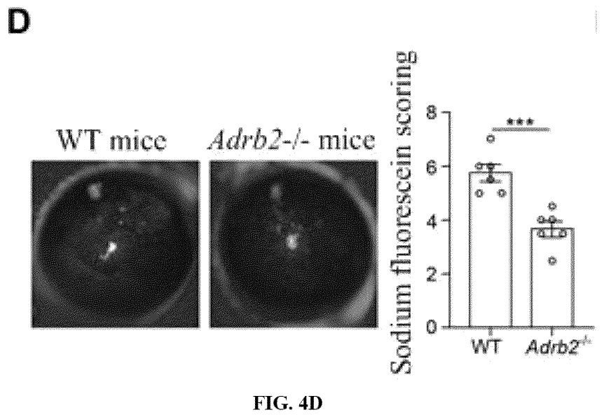

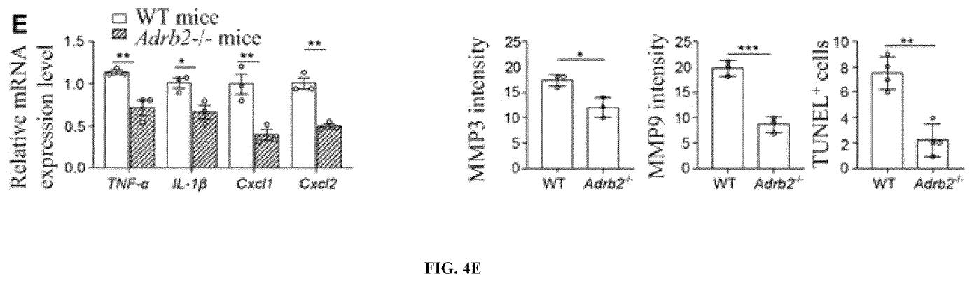

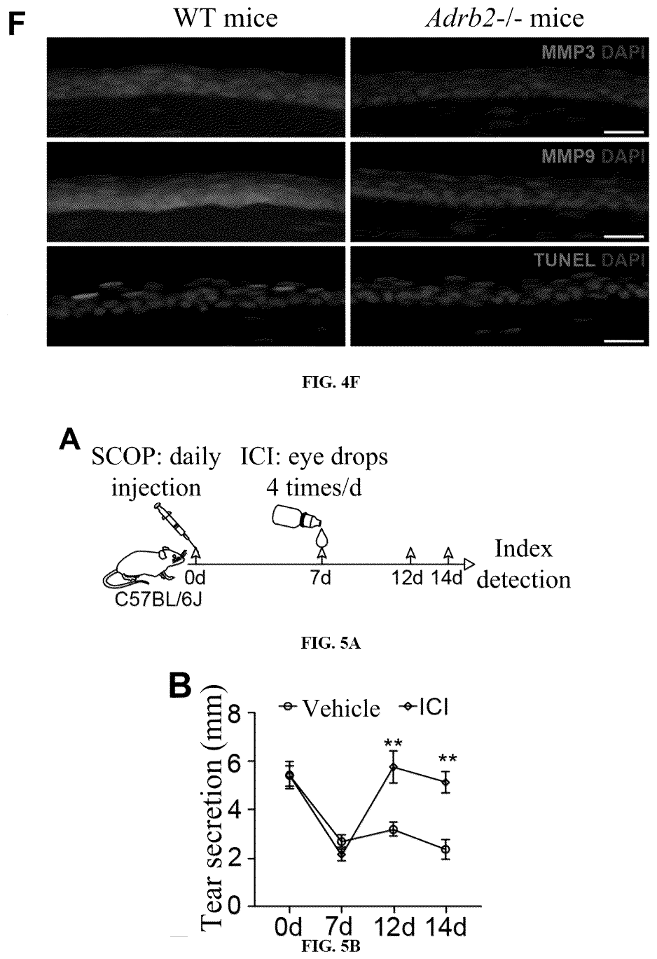

The SNS functions by releasing NE to interact with adrenergic receptors. To identify key receptors in DED pathogenesis, mRNA levels of adrenergic receptors in the cornea were assessed, revealing highest expression of Adrb2, followed by Adrb1 and Adra3 (FIG. 4A). To investigate the NE-Adrb2 (β2-AR) pathway, Adrb2-knockout (Adrb2−/−) mice and wild-type (WT) controls were compared for responses to NE-induced DED. Adrb2−/− mice exhibited significantly reduced corneal Adrb2 expression (FIG. 4B). Topical NE instillation reduced tear secretion in WT mice but not in Adrb2−/− mice (FIG. 4C). Sodium fluorescein scores showed marked improvement in Adrb2−/− corneas (FIG. 4D). Furthermore, corneal expression of TNF-α, IL-1β, Cxcl1, and Cxcl2 was significantly lower in Adrb2−/− mice versus WT (FIG. 4E). MMP3/MMP9 expression and epithelial apoptosis were also reduced in Adrb2−/− mice (FIG. 4F). These findings strongly support the direct role of the NE-Adrb2 pathway in DED pathogenesis.

### Topical Adrb2 Blocker Alleviating DED Symptoms

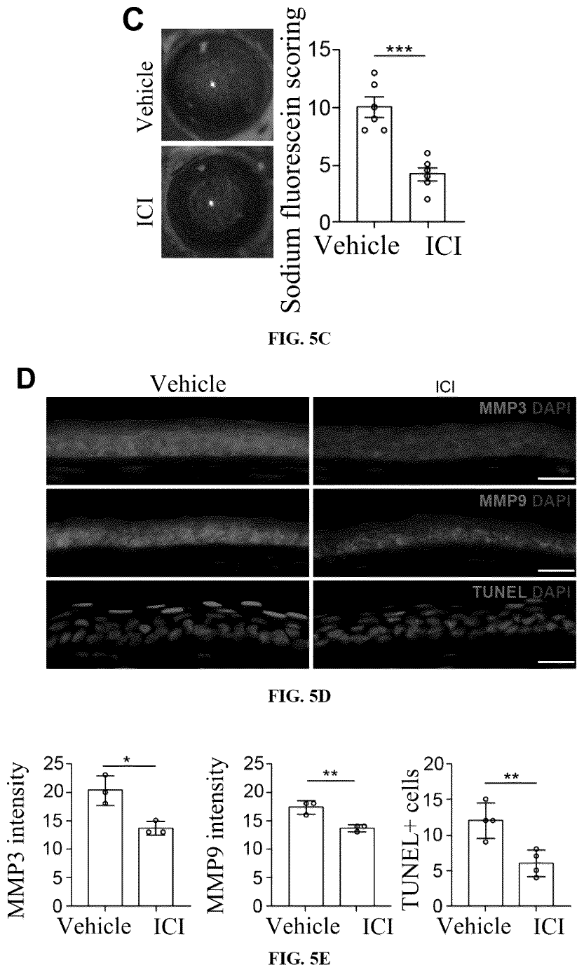

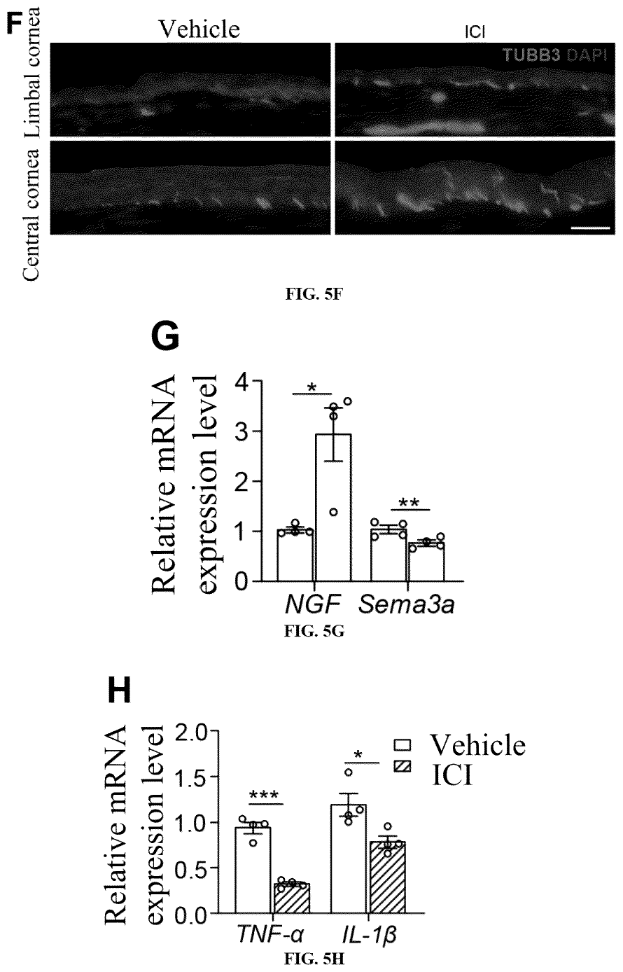

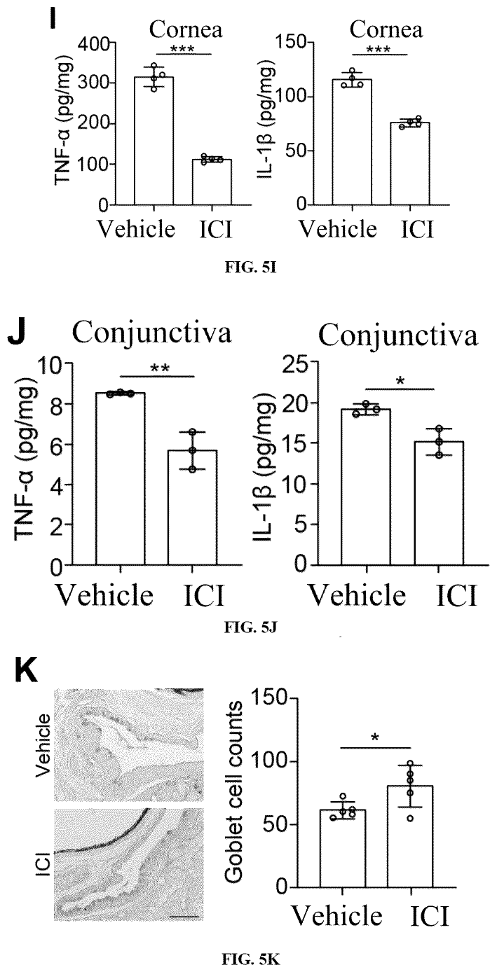

To validate NE-Adrb2 signaling pathway in DED, the β2-AR blocker ICI 118551 was topically administered after successful DED induction (day 7 post-scopolamine) (FIG. 5A). Tear secretion significantly recovered by day 5 of ICI 118551 treatment, showing a 2.2-fold increase versus controls (FIG. 5B). Corneal barrier integrity improved, with reduced sodium fluorescein scores after ICI 118551 treatment (FIG. 5C). Immunofluorescence confirmed ICI 118551 suppressed MMP3/MMP9 expression and decreased epithelial apoptosis (FIG. 5D and FIG. 5E). Given the critical role of corneal innervation in tear secretion (Efraim Y, et al. A synthetic tear protein resolves dry eye through promoting corneal nerve regeneration. Cell Rep. 2022; 40 (9): 111307), nerve fiber density was assessed via anti-Tubb3 staining. DED mice exhibited reduced nerve density in limbal and central corneal epithelium, while ICI 118551-treated mice showed restored innervation with fibers extending into superficial epithelium (FIG. 5F). ICI 118551 also upregulated mRNA of the neurotrophic factor Ngf in DED mice and downregulated repulsive axonal guidance cue Sema3a (FIG. 5G), indicating improved neural regeneration by Adrb2 blockade. RT-PCR and ELISA confirmed attenuated inflammation in cornea/conjunctiva post-ICI 118551 treatment (FIG. 5H and FIG. 5J). PAS staining revealed restored conjunctival goblet cell density (FIG. 5K). Collectively, these results demonstrated the therapeutic potential of Adrb2 antagonism for DED.

### Adrb2 Antagonism Modulating DED-Associated Inflammatory Responses

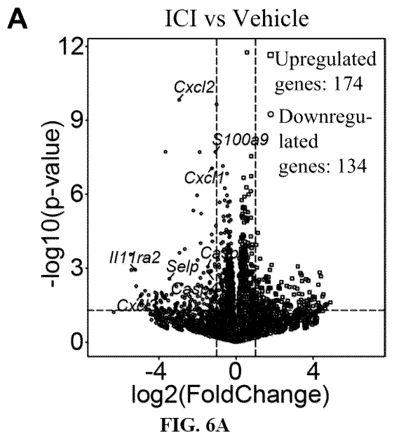

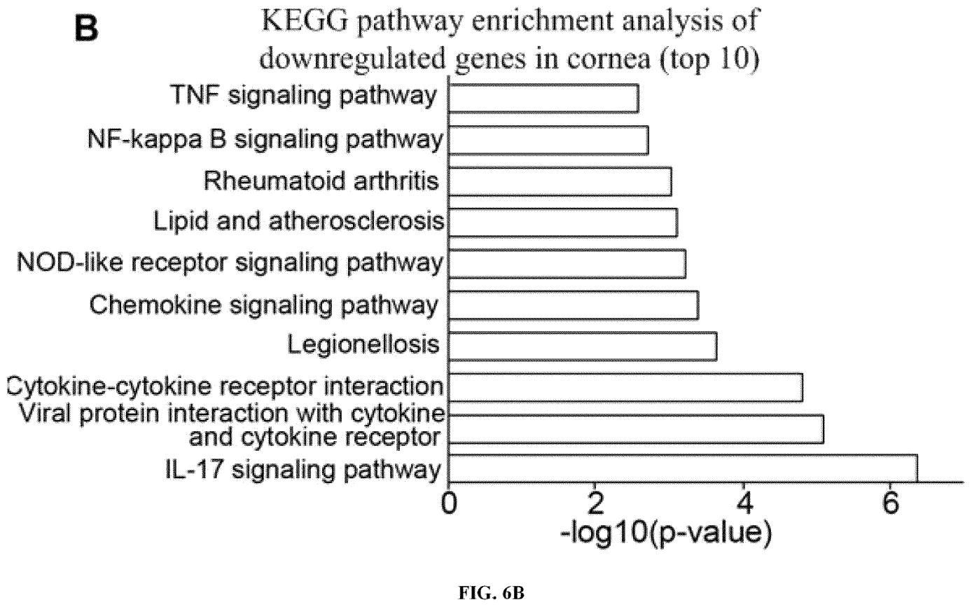

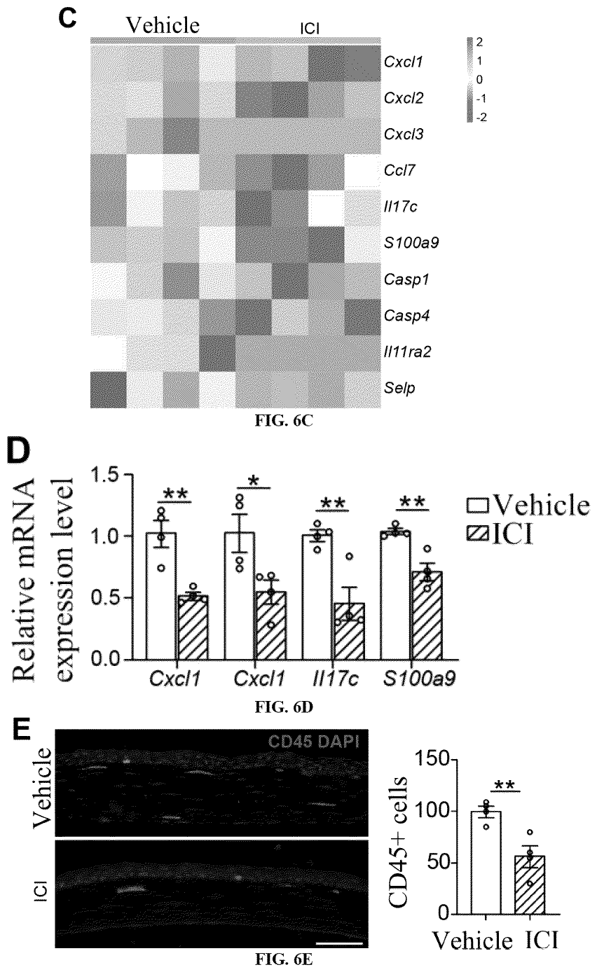

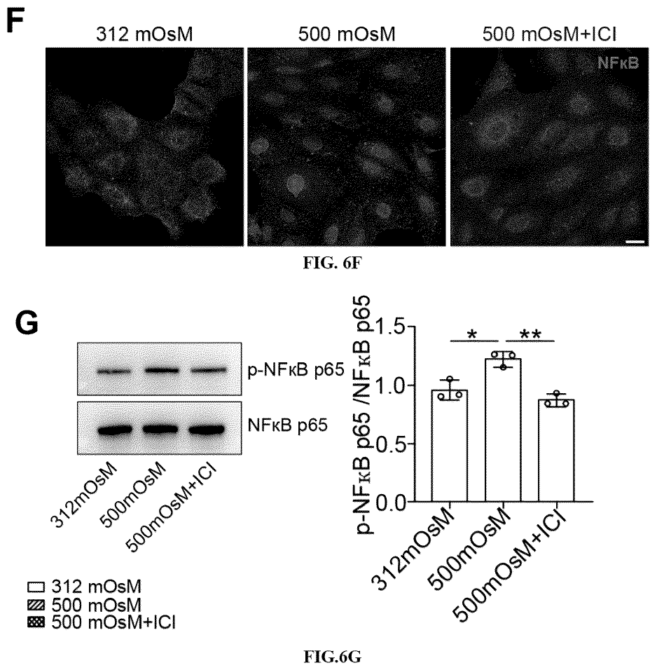

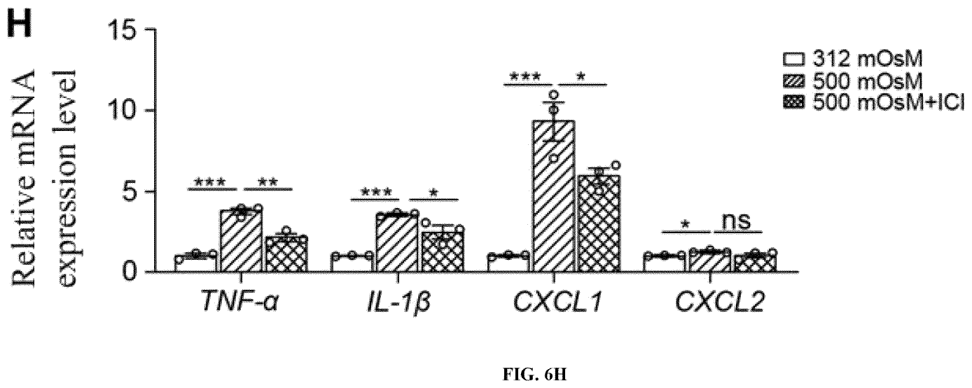

To elucidate the mechanism of Adrb2 blockade, RNA-seq analysis was conducted on corneas from ICI 118551- or vehicle-treated DED mice. ICI 118551 treatment induced upregulation of 174 genes and downregulation of 134 genes (FIG. 6A). KEGG pathway analysis revealed significant downregulation of canonical inflammatory pathways implicated in DED pathogenesis, including TNF, NF-κB, Chemokine, NOD-like receptor, and IL-17 signaling pathways (FIG. 6B). Heatmap analysis confirmed reduced expression of inflammation-related genes within these pathways (FIG. 6C). qRT-PCR validated significantly lower corneal expression of Cxcl1, Cxcl2, Il17c, and S100a9 in ICI 118551-treated versus vehicle groups (FIG. 6D). CD45 immunostaining demonstrated reduced inflammatory cell infiltration in ICI 118551-treated corneas (FIG. 6E). Focusing on the NF-κB signaling pathway, a key regulator of innate/adaptive immunity and cytokine production was studied (Zhang J, et al. Calcitriol, the Active Metabolite of Vitamin D (3), Inhibits Dry Eye Related Corneal Inflammation In Vivo and In Vitro. Ocul Immunol Inflamm. 2019; 27 (2): 257-265). The effect of ICI 118551 on NF-κB activation was examined in HCECs under hyperosmotic stress. Immunofluorescence showed hyperosmolarity-induced nuclear translocation of NF-κB, which was markedly suppressed by ICI 118551 (FIG. 6F). Hyperosmotic stress increased phospho-NF-κB levels, while ICI 118551 reduced this elevation by approximately 30% (FIG. 6G). Furthermore, ICI 118551 significantly decreased mRNA levels of TNF-α, IL-1β, and CXCL1 (but not CXCL2) under hyperosmotic conditions (FIG. 6H). These results demonstrated that Adrb2 blockade effectively mitigated DED-associated inflammation by suppressing key pathways including NF-κB.

### Example 2

### 20 μM to 1 mM ICI Ameliorating Dry Eye and Increasing Tear Secretion

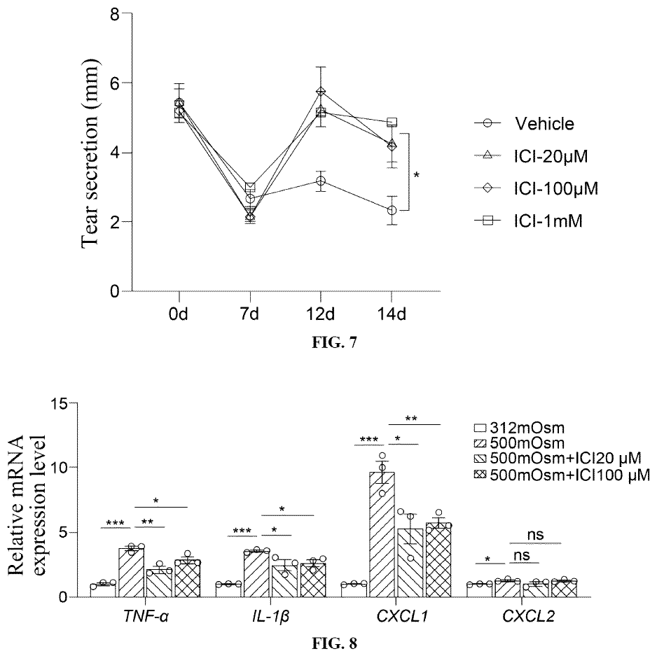

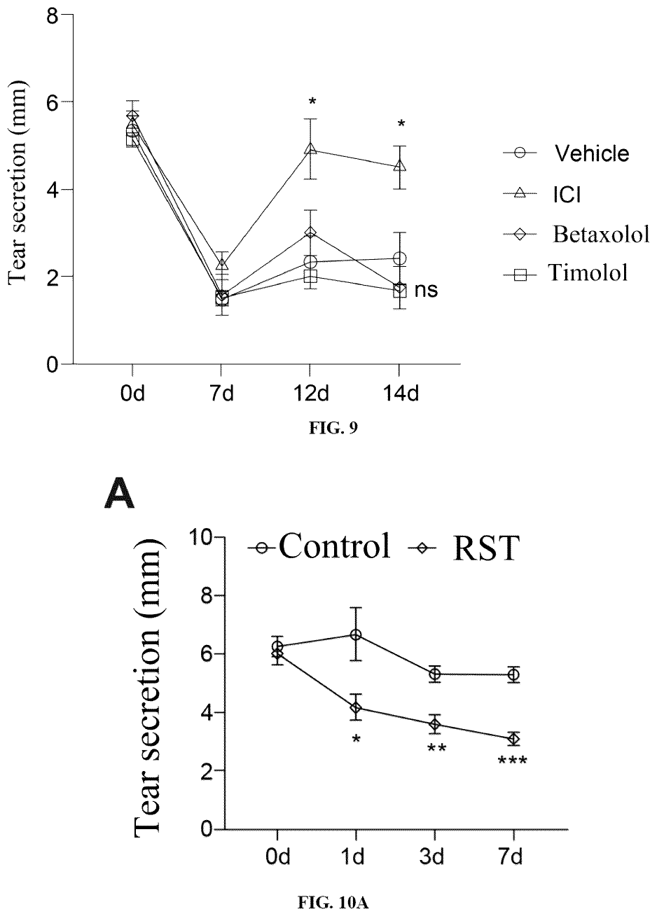

After successful DED induction (day 7 post-scopolamine), mice received topical β2-AR blocker ICI 118551 at varying concentrations (20 μM, 100 μm, 1 mM) (FIG. 7). By day 5 of treatment, all ICI 118551 groups exhibited statistically significant (p<0.05) recovery of tear volume compared to untreated DED controls. These results demonstrate that ICI 118551 within the 20 μM to 1 mM range effectively treats murine DED, thus restoring tear secretion and alleviating symptoms.

### Example 3

### In Vitro Validation: 20-100 μM ICI Reducing Hyperosmolarity-Induced Inflammatory Factor Expression in HCECs

HCECs were used to verify the effects of different concentrations of ICI 118551 on the expression of inflammatory factors under hyperosmotic stress. Treatment with 20-100 μM ICI 118551 significantly reduced mRNA levels of TNF-α, IL-1β, and CXCL2 under hyperosmotic conditions, while failing to affect CXCL1 expression. Hyperosmolarity is a common pathogenesis of DED. Hyperosmolarity can increase the expression of inflammatory factors in HCECs and aggravate ocular surface inflammation. HCECs treated with hyperosmolarity are generally used as an in vitro cell model for dry eye research. Experimental results demonstrated that treatment with 20-100 μM ICI 118551 significantly reduced mRNA levels of TNF-α, IL-1β, and CXCL2 under hyperosmotic stress, while exerting no significant effect on CXCL1 expression. Collectively, this reduction mitigated overall inflammatory burden in corneal epithelial cells, demonstrating that ICI 118551 alleviated ocular surface inflammation by suppressing hyperosmolarity-induced proinflammatory factor expression, thereby exerting therapeutic effects against DED.

### Example 4

Therapeutic Efficacy Comparison: Adrb2 Blocker Vs. Glaucoma Drug Betaxolol (β1 Blocker) and Timolol (β1&β2 Blocker)

Betaxolol and Timolol are Adrb blockers for glaucoma treatment that lower intraocular pressure (IOP). Betaxolol (0.25% to 0.5% eye drops) inhibits β-receptors in ciliary epithelium, reducing aqueous humor production. Timolol (0.25% to 0.5% eye drops) blocks β1/β2 receptors, decreasing aqueous humor production and potentially increasing outflow. NOTE: 20 μM ICI 118551=0.00062772% w/v represents 1/398 to 1/796 of clinical glaucoma drug concentrations (0.25% to 0.5%).

After DED induction (day 7 post-scopolamine), mice received: 20 μM ICI 118551; 0.25% Betaxolol (commercial glaucoma solution); 0.5% Timolol (commercial glaucoma solution) (FIG. 9). By day 5, ICI 118551 significantly restored tear secretion, whereas Betaxolol and Timolol showed no therapeutic effect.

### Example 5

### Anxiety/Depression-Associated Dry Eye Model

The restraint stress (RST) model simulated psychosocial stress linked to anxiety/depression (Sano, K. et al. Enriched environment alleviates stress-induced dry-eye through the BDNF axis. Sci. Rep. 9, 3422 (2019); Chiba, S. et al. Chronic restraint stress causes anxiety- and depression-like behaviors, downregulates glucocorticoid receptor expression, and attenuates glutamate release induced by brain-derived neurotrophic factor in the prefrontal cortex. Prog. Neuropsychopharmacol. Biol. Psychiatry. 39, 112-119 (2012). Female C57BL/6J mice (10-12 weeks) underwent 10-h daily restraint in 50 mL tubes for 7 d under desiccating stress, with controls having ad libitum access to food/water. Both groups of mice were placed in a dry stress environment for 7 d.

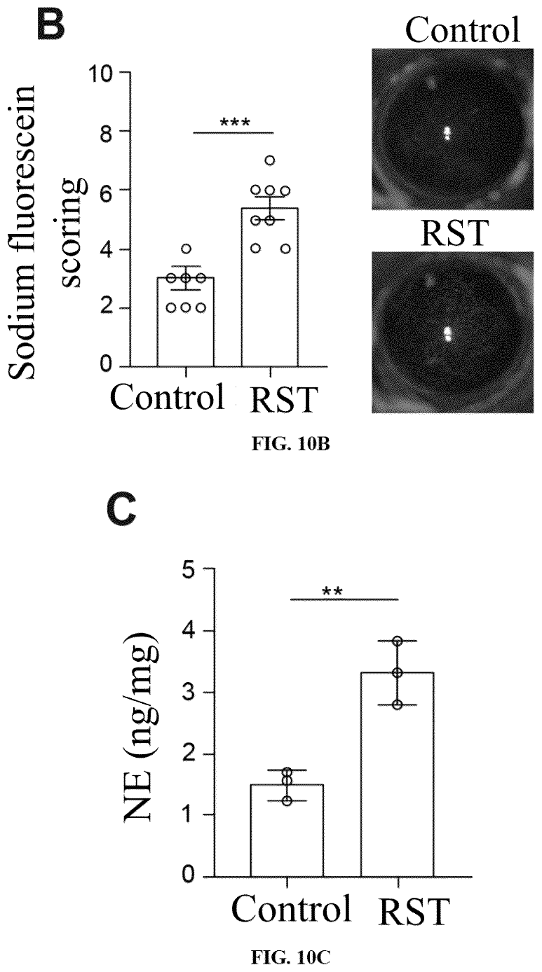

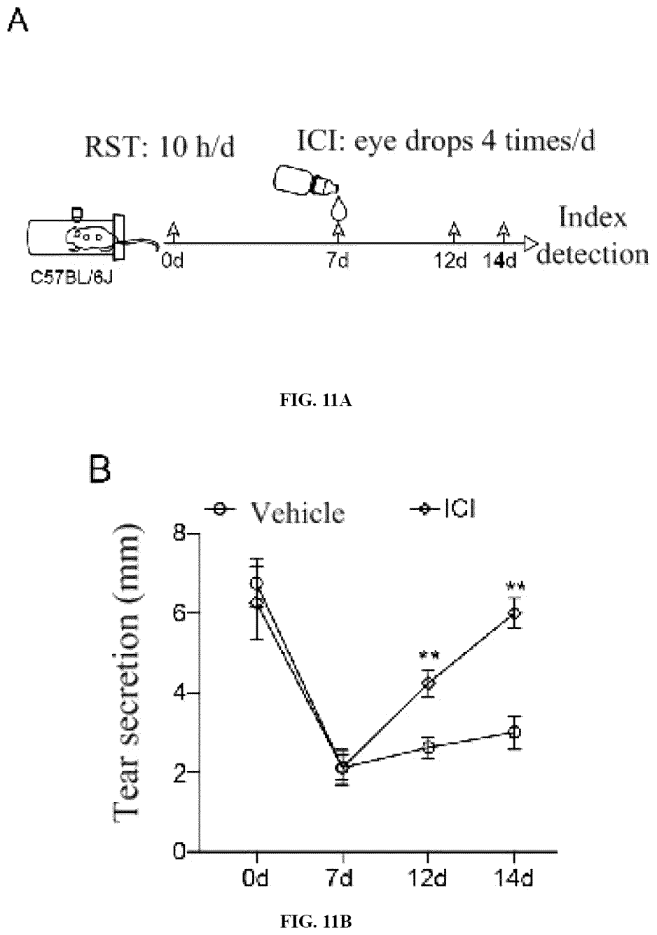

RST significantly reduced tear secretion, increased sodium fluorescein scores (showing DED symptoms), elevated corneal NE levels (FIG. 10).

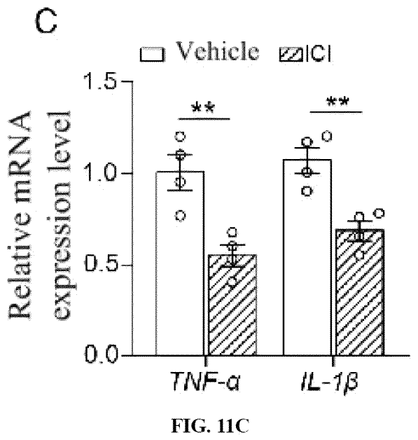

After 7 d of RST and the successful establishment of the dry eye model, the 12-AR blocker ICI 118551 was applied topically for eye drops (FIG. 11A). After 5 d of ICI 118551 eye drops treatment, the tear secretion of mice was significantly restored, increasing 1.6 times compared with the control group (FIG. 11B). ICI 118551 treatment also significantly reduced the mRNA expression levels of inflammatory factors TNF-α and IL-1β in the cornea of DED mice (FIG. 11C).

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A method for treating dry eye disease (DED), wherein the method comprises administering an agent targeting a norepinephrine-adrenergic receptor beta 2 (NE-Adrb2) signaling pathway to a subject in need thereof.

2. The method according to claim 1, wherein the agent targeting the NE-Adrb2 signaling pathway comprises an agent that blocks the NE-Adrb2 signaling pathway.

3. The method according to claim 1, wherein the agent targeting the NE-Adrb2 signaling pathway comprises an adrenergic receptor beta 2 (Adrb2) blocker.

4. A method for preparing a drug having at least one effect selected from the group consisting of i) to vi):
i) increasing tear secretion;
ii) improving a corneal epithelial barrier function;
iii) improving corneal innervation;
iv) reducing expression of a matrix metalloproteinase (MMP);
v) ameliorating inflammatory responses in cornea and conjunctiva; and
vi) restoring a conjunctival goblet cell density,
wherein the method comprises using an Adrb2 blocker as raw material to prepare the drug.

5. A method for downregulating an inflammation-associated pathway, wherein the method comprises administering an adrenergic receptor beta 2 (Adrb2) blocker to a subject in need thereof.

6. The method according to claim 3, wherein the Adrb2 blocker comprises ICI 118551.

7. The method according to claim 1, wherein the agent targeting the NE-Adrb2 signaling pathway comprises N-(2-chloroethyl)-N-ethyl-2-bromobenzylamine hydrochloride.

8. The method according to claim 1, wherein the agent targeting the NE-Adrb2 signaling pathway comprises adrenergic receptor beta (Adrb) blocker.

9. The method according to claim 1, wherein the DED is selected from the group consisting of aqueous-deficient DED and psychological stress-associated DED.

10. The method according to claim 1, wherein the agent targeting a norepinephrine-adrenergic receptor beta 2 (NE-Adrb2) signaling pathway is a topical ophthalmic formulation.

11. The method according to claim 4, wherein the Adrb2 blocker comprises ICI 118551.

12. The method according to claim 5, wherein the Adrb2 blocker comprises ICI 118551.

13. The method according to claim 2, wherein the DED is selected from the group consisting of aqueous-deficient DED and psychological stress-associated DED.

14. The method according to claim 3, wherein the DED is selected from the group consisting of aqueous-deficient DED and psychological stress-associated DED.

15. The method according to claim 7, wherein the DED is selected from the group consisting of aqueous-deficient DED and psychological stress-associated DED.

16. The method according to claim 8, wherein the DED is selected from the group consisting of aqueous-deficient DED and psychological stress-associated DED.

17. The method according to claim 2, wherein the agent targeting a norepinephrine-adrenergic receptor beta 2 (NE-Adrb2) signaling pathway is a topical ophthalmic formulation.

18. The method according to claim 3, wherein the agent targeting a norepinephrine-adrenergic receptor beta 2 (NE-Adrb2) signaling pathway is a topical ophthalmic formulation.

19. The method according to claim 4, wherein the drug is a topical ophthalmic formulation.

20. The method according to claim 5, wherein the adrenergic receptor beta 2 (Adrb2) blocker is a topical ophthalmic formulation.
